(19) United States
(12) Patent Application Publication
(10) Pub. No.: US 2011/0319774 A1
(43) Pub. Date: Dec. 29, 2011

(54) Kind of Blood Pressure Monitor Taking the Blood Pressure of Both Arms Simultaneously

(76) Inventor: Ben Jun Wu, Markham (CA)

(21) Appl. No.: 13/135,403

(22) Filed: Dec. 22, 2009

(51) Int. Cl. A61B 5/022

(52) U.S. Cl. 600/490

(57) ABSTRACT

This utility model provides a kind of blood pressure monitor to take the blood pressure of both arms simultaneously, including: power supply system, operation buttons, LCD monitor screen, plugholes for rubber tubes located on the LCD monitor screen, the rubber tube is plugged into the plughole and connected with the cuff of blood pressure monitor. It's characterized with dual plugholes for rubber tubes, dual rubber tubes and dual cuffs of blood pressure monitor. This utility model can monitor the blood pressure of both arms simultaneously and demonstrate the blood pressure of both arms simultaneously.

### FIELD OF THE INVENTION

This utility model involves a blood pressure monitor taking the blood pressure of both arms simultaneously.

### TECHNOLOGY BACKGROUND

Although the invention of blood pressure monitor has been a long history and it keeps renewing; there's has never been a blood pressure monitor that can take the blood pressure of both arms at the same time. This is because people never realize the importance of taking the blood pressure of both arms simultaneously. According to the Balance Theory of Traditional Chinese Medicine, the healthy human organs and their functions are in a balance status; once the balance status is broken, different kinds of disease appears. Blood pressure is an important performance reflecting the balance status of human organs and functioning. So traditional Chinese medicine relies on feeling the pulse and takes it as one of the main proofs to diagnose disease.

But to make a correct diagnosis by feeling the pulse is depending mainly on a doctor or medical physician's long time experience. And it can vary from different environment and different human bodies. It cannot digitize the diagnosis and the result is not precise.

The modern blood pressure monitor is working the same function as feeling the pulse in traditional Chinese medicine. By taking the blood pressure of both arms, we can find out if the human organs and functions are in balance status. However, the current method is applying on taking the blood pressure of one arm, then taking the blood pressure of another arm. Because of the difference of monitor time and the moving of patients, the data received is not exactly what it is showing concurrently. This utility model solves the problem by taking the blood pressure of both arms and demonstrating the blood pressure of both arms at the same time.

This utility model provides a blood pressure monitor which can take the blood pressure of both arms at the same time and demonstrate the blood pressure of both arms at the same time.

A kind of blood pressure monitor taking the blood pressure of both arms simultaneously includes: power supply system, operation buttons, LCD monitor screen, plugholes for rubber tubes located on the LCD monitor screen, the rubber tube is plugged into the plughole and connected with the cuff of blood pressure monitor. It's characterized with dual plugholes for rubber tubes, dual rubber tubes and dual cuffs of blood pressure monitor. This utility model can monitor the blood pressure of both arms simultaneously and demonstrate the blood pressure of both arms simultaneously.

Furthermore, there are dual LCD monitor screens for better convenience to observe the blood pressure data.

Furthermore, the dual LCD monitor screens arc installed side by side. It's for better convenience of symmetric installation and reasonable layout of blood pressure monitor; it saves the space and is portable to carry with.

### DESCRIPTION OF DRAWINGS

FIG. 1 is the structure layout of this utility model of a kind of blood pressure monitor taking the blood pressure of both arms simultaneously.

FIG. 2 is the structure layout of this utility model of a kind of blood pressure monitor taking the blood pressure of both arms simultaneously with two cuffs.

### OPERATIONS

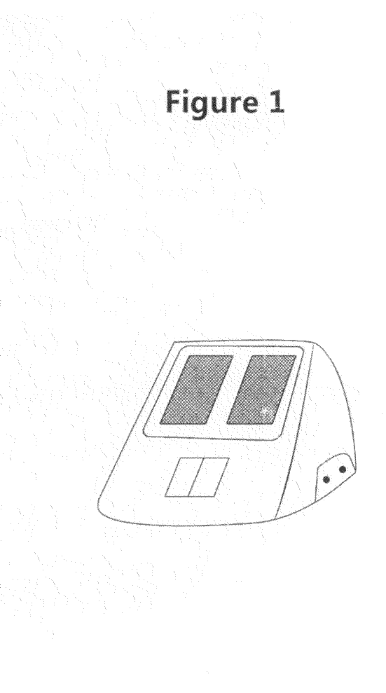

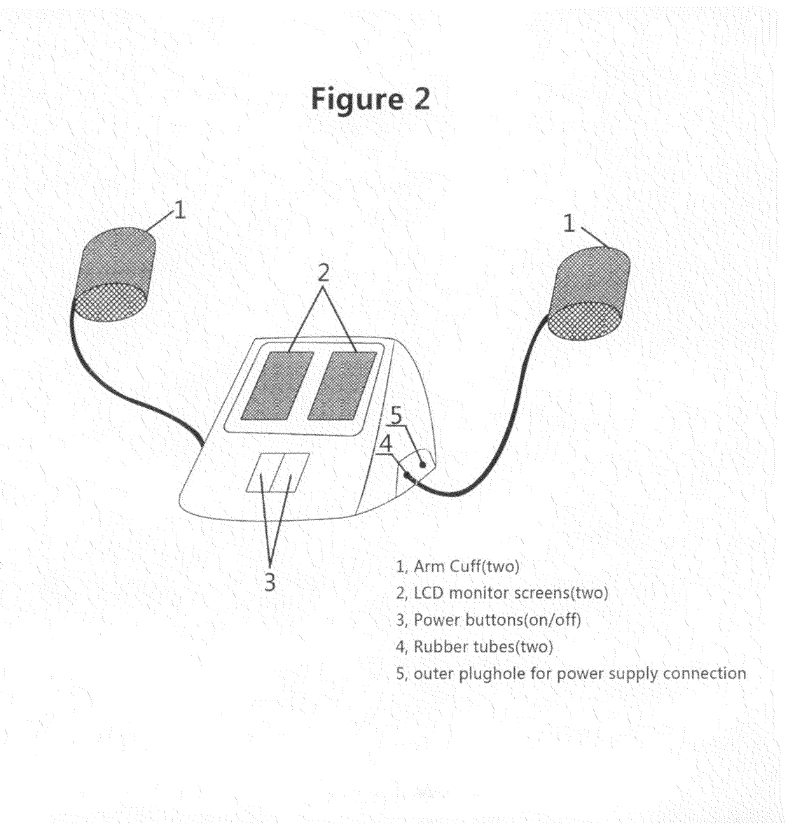

This utility model provides a kind of blood pressure monitor taking the blood pressure of both arms simultaneously which enables taking the blood pressure of both arms at the same time. A detailed description covering FIG. 1 and FIG. 2 is provided below for this utility model.

This kind of blood pressure monitor taking the blood pressure of both arms simultaneously includes: power supply system, operation buttons 3, two rubber tubes, two plugholes for rubber tubes 4, two cuffs for blood pressure monitor 1, and LCD monitor screen 2.

The power supply system can be the battery box and/or plughole of power supply. Specifically, there's an outer plughole 5 in the monitor for connecting power supply; in case of power off, the battery box can keep the blood pressure monitor working. A battery box is designed in the back of the blood pressure monitor.

Operation buttons 3 includes the Start button and Stop button for starting and ending the working process of the blood pressure monitor.

Plugholes for rubber tubes 4 are installed on the LCD monitor screen 2, the rubber tubes are plugged into the plugholes and connected with the cuffs of blood pressure monitor. Because there are two rubber tubes and two rubber tube plugholes, accordingly they are connected with two cuffs of blood pressure monitor for taking the blood pressure of both arms.

This kind of blood pressure monitor taking the blood pressure of both arms simultaneously 1 is used to take the blood pressure of both arms; when applying to use, the cuffs are wrapped over the left arm and the right arm respectively; the cuff can boost the pressure automatically.

LCD monitor screen 2, will demonstrate the blood pressure of both arms simultaneously. The LCD monitor screen is connected to an end of each of the cuffs, so the LCD monitor screen will demonstrate the blood pressure data of both arms. Furthermore, two separate LCD monitor screens can be installed; every single LCD monitor screen is connected to a single cuff of blood pressure monitor. After taking the blood pressure of both arms, the blood pressure data will be shown on two LCD monitor screens. The two monitor screens can be installed side by side, or called symmetric installation, as shown in FIG. 1. It's for better convenience of the doctor or medical physician to observe the blood pressure data of two arms and to study the balance status of the patient's organs and functioning.

1. A kind of blood pressure monitor taking the blood pressure of both arms simultaneously includes: power supply system, operation buttons, LCD monitor screen, plughole for rubber tubes located on the LCD monitor screen, the rubber tube is plugged into the plughole and connected with the cuff of blood pressure monitor. It's characterized with dual plugholes for rubber tubes, dual rubber tubes and dual cuffs of blood pressure.

2. According to claim 1, a kind of blood pressure monitor taking the blood pressure of both arms simultaneously is characterized with dual LCD monitor screens.

3. According to claim 2, a kind of blood pressure monitor taking the blood pressure of both arms simultaneously is characterized with dual LCD monitor screens installed side by side, or called symmetric installation.
